(19) United States
(12) Patent Application Publication
(10) Pub. No.: US 2007/0118402 A1
(43) Pub. Date: May 24, 2007

(54) System and Method for Consumer/Patient Flushot Reservation

(76) Inventor: John E. Deneen, Villanova, PA (US)

Correspondence Address: Connolly Bove Lodge & Hutz LLP, 1007 North Orange Street, P.O. Box 2207, Wilmington, DE, 19899, US

(21) Appl. No.: 11/468,987

(22) Filed: Aug. 31, 2006

Related U.S. Application Data
(63) Continuation-in-part of application No. 11/219,111, filed on Sep. 2, 2005.

(51) Int. Cl. G06Q 50/00

(52) U.S. Cl. 705/2

(57) ABSTRACT

This invention relates to the field of vaccine management and, in particular to a system and method for reserving a dose of influenza vaccine. The system for reserving a flu-shot comprises a user interface and a matching node. The method comprises receiving a flu shot reservation from a user and matching the reservation to a health care provider.

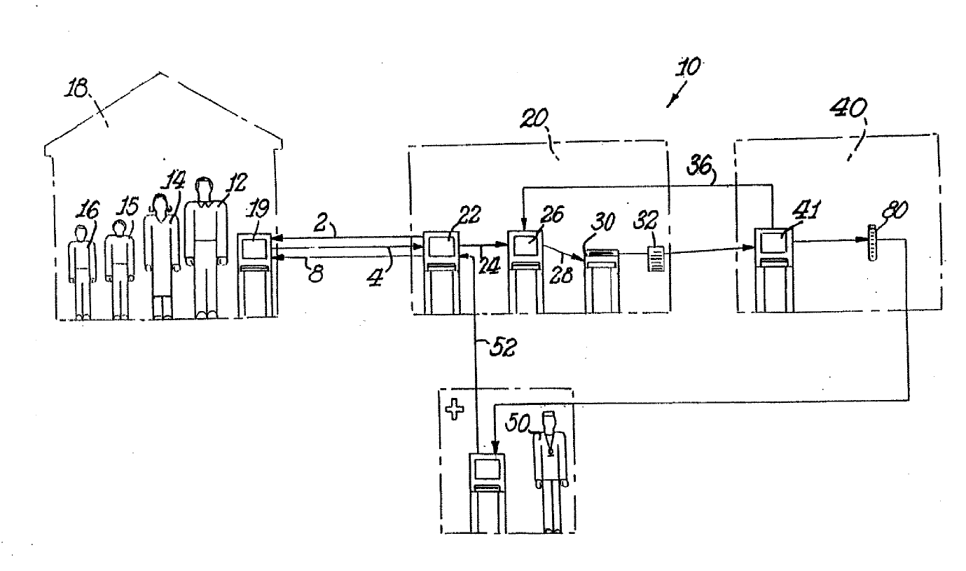

### RELATED APPLICATIONS

This application claims benefit of priority to U.S. application Ser. No. 11/219,111, filed on Sep. 2, 2005 and U.S. application Ser. No. 11/418,987.

### FIELD OF THE INVENTION

This invention relates to the field of vaccine supply management. Specifically, the invention relates to consumer/patient participation in flu-shot reservations to refine vaccine management.

### BACKGROUND OF THE INVENTION

Vaccines exist for a host of diseases. Vaccine development for specific diseases is identified by the medical community and government health agencies. Issues considered in identification include difficulty and cost of post-infection treatment, potential loss of life and ability to manufacture an efficacious vaccine. The medical community and government work closely with vaccine manufacturers to develop and produce sufficient supplies of vaccine.

Viral influenza is one disease identified by public health authorities for vaccination. In the United States, influenza causes on average in excess of 20,000 deaths and 110,000 hospitalizations annually. The impact of influenza on humans depends on (1) the degree of protective immunity in the population, (2) the influenza strain's ability to be passed from person-to-person, and (3) influenza strain virulence. Of these three aspects of the disease, protective immunity is addressed by a vaccine program.

Vaccination is a controlled exposure to an immune system with an innocuous component of a pathogenic agent in a disease. The vaccination exposure prompts the immune system to react by generating memory cells. Memory cells generate disease specific antibodies to fight off future infections by the same pathogenic agent. As a result, memory cells represent “immunity resistance.” For a vaccine to be effective, it is based upon a component of the disease's pathogenic agent called an antigen. Antigens are constant structures across all strains and variants a pathogenic agent may possess. An antigen must also elicit a strong immune response from the human immune system. A stronger immune response creates better immune resistance both for fighting infection and for retention of immune resistance over time.

Vaccination programs usually target children to establish long term resistance. Many recipients need only a single vaccination per disease to establish immunity. Under certain circumstances, a booster vaccination may be administered. The variables dictating a single vaccination versus booster vaccination involve the nature of the disease/pathogen, the frequency of the recipient's exposure and/or the strength of the immune response to the vaccine. Therefore, under most circumstances, vaccine supply management is straightforward. A single dose of vaccine is given to a subset of the population on a set schedule over several years with a few members requiring boosters several years later. Health care providers and manufactures can accurately predict demand and match it with optimal production capacity. However, viral influenza does not fit into this vaccine supply management model.

Viral influenza in humans has two sub-types, influenza type A and influenza type B. Influenza type A viruses are further classified into subtypes on the basis of two surface antigens: hemagglutinin (H) and neuraminidase (N). Both types A and B viruses undergo continual antigenic drift resulting in the constant emergence of new strains or variants. Antibody against one type or subtype confers little or no protection against another influenza type or subtype. In addition to antigenic drift, influenza type A viruses can undergo a more dramatic and abrupt antigenic shift. Antigenic shift occurs when a virus belonging to a new influenza A subtype bearing either a novel hemagglutinin protein or novel hemagglutinin and neuraminidase proteins begins circulating among people. Antigenic shift results from an exchange of genetic material between two different influenza viruses and/or changes in an influenza virus that previously infected only non-human animals that now allows the virus to be transmitted to people, i.e. to jump a species barrier.

The constant development of antigenic variants through antigenic drift prevents humans from establishing long term immunity to influenza. Humans thus experience reoccurring influenza infections. This constant antigenic change is why influenza vaccine must be updated annually. Annual influenza vaccination is the most effective way to counter the antigenic drift of viral influenza.

Viral influenza in the United States peaks from December to March. This “flu season” starts and ends a few weeks before and after this peak time frame. The annual viral influenza vaccine formulation is updated early each year, based on trends observed through the most recent flu season. Vaccine composition is specifically based upon an extrapolation of which strains of influenza will most likely circulate during the next flu season. The immune response takes several weeks to reach an optimum after injection. Accordingly, vaccination should occur in advance of flu season, i.e., in the September to October time frame. Production of a sufficient amount of vaccine must occur in a very narrow time frame of a few months. The importance of production of a sufficient amount of vaccine was illustrated most recently in 2004. In 2004, half of the United States vaccine supply for a normal flu season was removed from the market due to production problems. The result was a rationing of vaccine. This led to increased potential for an above average impact of influenza upon the public health as well as an increased potential for an influenza pandemic.

During the 20^{th} century, pandemics occurred in 1918, 1957, and 1968, and caused widespread illness, death, and social disruption. The 1918-1919 Spanish Influenza pandemic is the catastrophe against which all modern pandemics are measured. Twenty million people died world-wide, 500,000 in the United States alone. Twenty to forty percent (20%-40%) of the world's human population became severely ill. The Centers for Disease Control estimates the next influenza pandemic could kill 89,000-207,000, hospitalize 314,000-733,000 and infect 18-42 million in the United States alone. Absent a sufficient supply of vaccine, any pandemic will likely exceed these numbers.

Therefore, managing the logistical challenges of producing sufficient influenza vaccine is critical. Manufacturers have responded by using the internet to assist in the ordering and management of influenza vaccine. Currently, all four United States vaccine manufacturers utilize the internet to manage vaccine orders from their customers. However, because vaccine is a prescribed medicine, from the perspective of the manufacturer, the customer is invariably a health care provider. Unlike childhood scheduled vaccinations, health care providers have no guide to accurately project viral influenza vaccine needs. However, ordering vaccine doses for every single patient is financially risky for the health care provider. The health care provider must pay out of pocket in advance for all doses. The health care provider becomes open to a significant financial vulnerability if he orders more vaccine than is needed. Ordering less avoids financial risk, but exposes patients to the preventable medical risks of viral influenza.

The predicative problems at the health care provider level are magnified at the manufacturer level once all the inherently inaccurate orders are aggregated and production predictions are made based upon speculative, indirect consumer/recipient demand. In the event of an influenza pandemic, the entire vaccine production capacity would be overwhelmed with a sudden, immediate need for vaccine for, in theory, the earth's entire human population. The resulting loss of life would be incalculable and completely unnecessary.

What is needed is a method and/or system permitting accurate production projections for vaccine manufacturers to insure that an accurate and adequate supply of vaccine is produced. More specifically, what is needed is a method and/or system permitting direct consumer demand determinations of vaccine production projections without violating medicament prescriptions laws. Additionally, in the event of an influenza pandemic, a substantial ready reserve of production capacity is needed to meet sudden, immediate demand. A demand that would instantly overwhelm industrial capacity built around annualized demand.

### SUMMARY OF THE INVENTION

The present invention includes a method and system for reserving a flu shot. In a preferred embodiment, the invention is a method for reserving a flu-shot, the method comprising: receiving a flu-shot reservation from a user/prospective recipient; and matching the reservation to a health care provider. The flu-shot reservation can be received by an internet connection. In addition, the reservation can be received via a telephone connection (e.g., a phone call) or by postal service. In a most preferred embodiment, the reservation is received over an internet connection.

The receipt of a flu-shot reservation may be accompanied by payment for the reservation. Payment may be received contemporaneously with the reservation or non-contemporaneously. Payment can be received via a variety of methods, including check, money order, cash, or credit card, debit card or wire transfer. Payment can also be received via direct withdrawal from a user's bank account(s) such as by wire transfer. In addition, the user can provide credit card information to a webpage. Systems for receiving credit card information are known and used on well-known websites such as Amazon.com. In another preferred embodiment, payment is received in a secure manner. Secure web-pages for receiving credit card payments are well-known in the art. In addition to receiving payment concurrent with the reservation, an annual auto renewal authorization can be designated by the user or made a default setting for subsequent annual payments and reservations.

In a further preferred embodiment, the step of receiving the flu-shot reservation comprises receiving additional data parameters. These include: (a) number of flu-shot(s), (b) name of recipient(s), (c) address of recipient(s), (d) health care provider preference(s), and (e) insurance provider(s). The data parameters need not be specific to the person making the reservation. By way of example, the reservation may be made by a head of household for one or more members of his or her family. In a further preferred embodiment, the data parameters are received in a secure manner.

The flu-shot reservations may be received in a reservation receiving center. The reservation receiving center may include a computer, a server, a series of servers and/or computers, an office, or multiple offices. In a preferred embodiment, the reservation receiving center may be a server, a telephone call center, a mailroom or a combination thereof. The servers and/or computers need not all be at the same physical location.

The method of reserving a flu-shot may further comprise prompting the user to request a flu-shot reservation. The prompting may include making a reservation for a person other than the user. Prompting can occur in a variety of formats, and may include an advertisement or solicitation. A prompt can be an electronic mail message.

The method of reserving a flu shot may include a step of consolidating flu-shot reservation requests for a health care provider into a consolidated flu-shot order. The consolidation step may be achieved in a variety of manners. These include compilation by hand, by use of a computer and/or server, or by use of a bar code scanner attached to a computer. Computer programs useful in consolidating the orders may include an Excel spreadsheet. These consolidated flu shot reservations may form the basis for an order placed to a flu shot manufacturer.

The method of reserving a flu shot may further comprise a step of communicating a consolidated flu-shot order to a flu-shot manufacturer. Communicating the flu-shot reservation may be accomplished by a wide variety of methods including an internet connection, a telephone, electronic mail, facsimile, regular mail, or courier service.

The method may further comprise a step of prompting a user to make an appointment with a health care provider matched to said user to receive the flu-shot. The step of prompting the user to make an appointment to receive a flu-shot may include placing a telephone call (e.g. via person or pre-recorded), sending an electronic mail, sending a letter, or a combination thereof. The step of matching the user to a health care provider comprises matching a data parameter of a health care provider with a data parameter of the reservation. In a most preferred embodiment, this could be matching the name of the physician with the name of the primary care physician identified by the user.

The method may further comprise a step of communicating health care provider receipt of a flu-shot order to the user. The step of communicating health care provider receipt of a flu-shot order may include placing a telephone call, sending an electronic mail, sending a letter or a combination thereof.

As used herein, the term “user” includes any individual(s) or organization(s) that interface with the invention. A user may include a patient, a representative of a patient (e.g., a parent of a child patient or the head of a household or the care-taker of an elderly or ill patient, a business, employer or entity).

As used herein, the phrase “health care provider” includes any individual, entity, organization, association or group of individuals that facilitates the provision of health care services to a user. The health care provider need not have any relationship or affiliation with the health care industry. A health care provider may include an employer or business, a volunteer organization or individual, an insurance company, a federal, state or local government agency, entity, corporation or affiliate, a charitable organization, a medically trained individual(s) (physician, nurse, paramedic, pharmacist, or assistant thereof) a hospital, a clinic, a nursing home, hospice or hostel or individual agent of any of the entities listed.

As used herein, the term “intermediary” includes any individual, entity, organization, association or group of individuals that facilitates the provision of health care services to a user. The intermediary need not have any relationship or affiliation with the health care industry. A intermediary may include an employer or business, a volunteer organization, an insurance company, a federal, state or local government agency, entity, corporation or affiliate, a charitable organization, entity, corporation or affiliate, a medically trained individual(s) (physician, nurse, paramedic, pharmacist, or assistant thereof) a hospital, a clinic, a nursing home, hospice or hostel or individual agent of any of the entities listed.

The invention may also be practiced by using a system for making flu shot reservations. Thus, the system for reserving a flu-shot comprises a means for receiving a flu-shot reservation from a user and a means for matching the reservation to a health care provider. The means for receiving the flu-shot reservation includes an internet connection, a telephone, a mail room, a post office box, a webpage, or an electronic mail account. The means for matching the reservation to a health care provider includes a filing cabinet with physician names included on files, a computer program, and phone logs.

In a preferred embodiment, the means for receiving the flu-shot reservation further comprises a means for receiving payment. The means for receiving payment includes a cash register, a credit card reader, a bar code scanner, a wire transfer instruction to a bank, and a webpage for entry of credit card information. In a preferred embodiment, payment is received in a secure manner. Secure webpages for receiving credit card payments are well-known in the art.

In a preferred embodiment, the means for receiving the flu-shot reservation further comprises a means for designated an annual auto renewal and/or payment. The means for receiving auto renewal includes a check box on a form (paper or electronic e.g. in an e-mail or a GUI/webpage), a clause on a form (paper or electronic), a pull down in a GUI/webpage, etc. In a preferred embodiment, auto renewal is a default option and upon auto renewal payment is concurrently submitted with the flu-shot reservation.

In another embodiment, the means for receiving the flu-shot reservation further comprises a means for designated an annual pandemic influenza reservation option contract. The means for receiving the annual pandemic influenza option contract includes a check box on a form (paper or electronic e.g. in an e-mail or a GUI/webpage), a clause on a form (paper or electronic), a pull down in a GUI/webpage, etc. The annual pandemic influenza option contract can be paid for utilizing the same means for receiving payment. The annual pandemic influenza option contract can also be renewed automatically. In a preferred embodiment, auto renewal is a default option and upon auto renewal of the option contract payment is concurrently submitted.

A different embodiment of the invention utilizes the flu-shot reservation system but instead of reserving an annual flu-shot, a user can opt to enter into only an annual pandemic influenza option contract.

In a preferred embodiment, the means for receiving provides for receipt of one or more of the following data parameters: (a) number of flu-shot(s), (b) name of recipient(s), (c) address of recipient(s), (d) health care provider preference(s), and (e) insurance provider(s). It is preferred that the means for receiving is secure.

The system may further comprise means for storing the user's flu-shot reservation request in a reservation receiving center. The means for storing may include a filing cabinet, a computer program, a computer or computers, a spreadsheet, a server, or a series of servers.

The system may further comprise a means for prompting the user to request a flu-shot reservation. The means for prompting includes a post card, a bill board advertisement, an electronic mail message, a telephone contact, or a webpage.

The system may further comprise a means for consolidating flu-shot reservation requests for a health care provider into a consolidated flu-shot order. The means for consolidating include a ledger, filing cabinet, a spreadsheet, a server, a computer, or a series of servers or computers. These consolidated flu shot reservations may form the basis for an order placed to a flu shot manufacturer.

The system further comprises a means for communicating a consolidated flu-shot order to a flu-shot manufacturer. The means for communicating includes a telephone, a fax machine, an electronic mail account, an electronic mail message, a post card, and a purchase order.

The system may further comprise a means for prompting a user to make an appointment with a health care provider matched to said user to receive the flu-shot. The means for prompting includes a telephone contact, an electronic mail message, a flyer, a billboard advertisement, a letter, a webpage, or a combination thereof.

The system may further comprise a means for matching the user to a health care provider. The means for matching includes a filing cabinet, a ledger, a computer program, a spreadsheet, a database, a computer, a server, or a series of computers or servers.

The system further comprises a means for communicating health care provider receipt of a flu-shot order to the user. The means for communicating to the health care provider receipt of a flu-shot order comprises a telephone contact, an electronic mail message, a letter, or a combination thereof.

The system of the present invention may include a user interface and a matching node for matching a user to a health care provider. The user interface may be a website, a telephone, a mail order form, or an office. In a preferred embodiment, the user interface is capable of receiving data from the user. In a most preferred embodiment, the data can be one or more of the following: number of flu-shot(s), name of recipients(s), address of recipient(s), health care provider preference(s), and insurance provider(s). In a most preferred embodiment, the user interface is capable of receiving payment from the user.

In a preferred embodiment, the matching node comprises a database of health care providers and is capable of matching a data parameter of the user to a data parameter of a healthcare provider.

The system preferably also includes a reservation receiving center for receiving a flu-shot reservation. The reservation receiving center comprises a server, a telephone call center, a mailroom or a combination thereof.

The system further comprises a consolidation node for consolidating flu-shot reservation requests for a health care provider into a consolidated flu-shot order. The consolidation node comprises a database of health care providers, in communication with the matching node, to consolidate flu-shot reservations matched to a healthcare provider into a consolidated flu-shot order. In a preferred embodiment, the consolidation node is capable of sending the consolidated flu-shot order to the flu-shot manufacturer.

The system may also include a communications network permitting communication between the user interface and the matching node. The communications network comprises a telephone network, a computer network, a mail delivery network, a satellite network, a wireless network, an electronic mail network, an internet connection or a combination thereof. The communications network is preferably secure.

The system is preferably capable of prompting the user to make an appointment for a flu-shot by telephone, electronic mail, a letter or a combination thereof.

### BRIEF DESCRIPTION OF THE DRAWINGS

Illustrates a system for reserving flu shots according to the present invention.

### DETAILED DESCRIPTION OF THE INVENTION

The embodiments of the present invention will now be discussed. The particulars shown herein are by way of example and for illustrative discussion of the embodiments of the present invention.

The sole figure of the patent drawing shows a system 10 for making a flu-shot reservation and/or pandemic influenza option contract in accordance with the present invention. User 12 is a male head of household residing in residence 18. User 12 has a wife 14 and two sons, 15 and 16.

Reservation/pandemic option contract receiving center 20 is a commercial building or a mobile unit. Reservation/pandemic option contract receiving center 20 includes a first server 22 that hosts a webpage dedicated to receive flu-shot reservations. The webpage may have the internet address www.flushots.com or a variant thereof. In the months preceding flu season, first server 22 sends a prompt 2 in the form of an electronic e-mail to user 12 computer 19 encouraging user 12 to make a flu-shot reservation and/or a pandemic influenza option contract for himself and his family. In response to prompt 2, user 12 uses computer 19 and logs onto a website hosted by first server 22.

User 12 identifies the names and addresses of the intended flu shot recipients in a flu shot reservation and/or pandemic influenza option contract 4, and further identifies family physician 50 as the physician or health care provider to whom the flu-shots should be sent.

First server 22 transmits the reservation and/or pandemic influenza option contract to second server 26 via connection 24. In this embodiment, separate servers are shown, but it should be understood that a single server or computer could perform all of the steps necessary to effectuate the system. At an appropriate time prior to flu season, second server 26 stores the flu shot reservation and/or pandemic influenza option contract 4. Second server 26 contains software capable of consolidating all flu shot orders for family physician 50 into a consolidated flu shot order. Consolidated flu shot order is transmitted to printer 30 via connection 28. Printer 30 prints out the consolidated flu shot order 32 in a purchase order format on behalf of family physician 50. Consolidated flu shot order 32 is transmitted to flu shot manufacturer 40 server station 41 via the United States postal system.

Flu shot manufacturer 40 receives order 32 and manufacturers an amount of flu vaccine 80 sufficient to fill order 32. Upon shipping flu vaccine 80 to family physician 50, flu shot manufacturer 40 sends a notice 36 from server 41 to server 26 via the United States postal system, e-mail, express mail, etc. to reservation receiving center.

In a further embodiment also shown in the patent drawing, family physician 50 sends a notice 52 to reservation receiving center 20 that he has received flu vaccine 80. Reservation receiving center 20 sends confirmation to user 12 via prompt 8 that the physician has received the flu vaccine 80. Prompt 8 is in the form of an electronic mail.

User 12 makes an appointment or several appointments for his family with family physician 50. Flu vaccine 80 is administered to user 12 and his family at said appointment.

Reservation receiving center 20 may provide a series of forms that allow healthcare providers to register to participate in the system. These are the providers that will be available to patients as they place their order, but the system may also include providers who do not register.

Once orders are consolidated and delivered to the health care provider, the system may transfer partial funds collected from the patient at the order to the health care provider at one or more appropriate intervals. These include dose ordering, shot dispensing, order and cancellation.

For a health care provider who prefers to outsource vaccine orders, the system may consolidate reservations and place an order with vaccine distributor(s)/manufacturer(s) at the appropriate time of the season.

In the event of an influenza pandemic the annual pandemic influenza option contracts operate to rapidly deliver vaccine. The annual pandemic influenza option contract would be non-refundable in the event a pandemic does not occur. The revenue generated from the option contracts incentivizes manufactures to establish additional vaccine research, development and production capacity. The additional production capacity from year to year may not be necessary. However, when the pandemic influenza event occurs manufactures would be able to respond rapidly. The rapid response would insure that life saving vaccine is produced and distributed rapidly, resulting in the significant reduction of influenza deaths. The annual pandemic influenza option contract also precludes the need for government intervention in vaccine production and distribution. Preventing governmental intervention prevents unnecessary complications and delays during the pandemic event.

Methods for receiving flu-shot reservations from a user and matching said reservations to a health-care provider can include a web-based solution, with functionality supported through an application server and database server hosted at a secured data center with back-ups and a recovery system.

A web site used in the present invention may comprise html documents, images, Java applications, JavaScript, and CSS files (or the equivalent thereof) and will integrate with an application server (for example, Java-based) and database server (for example, Oracle based).

Security may be required due to the nature of the patient information related to the reservation as well as the quantity of such information and healthcare provider information. Reservations may be entered through a series of secure forms on a web site, either directly by patients, or by agents acting on behalf of the system (e.g., taking orders from patients via a call center). Security may be provided through industry standard encryption, such as 128-bit SSL encryption certificate, and may be used in association with all forms requesting private information, as well as in the storage of appropriate data in the database.

Payment may be facilitated through a secure online payment system configured to accept credit card transactions. The system may include a basic shopping cart payment gateway for credit card verification and merchant account for deposit of funds. Patient credit card information or other private data may be protected through SSL encryption and encrypted storage in the database. The payment system may also allow payments through PayPal® or similar e-money provider.

The reservation receiving center may charge a processing fee that may immediately be deducted from patient payment account and transferred to an operational account. The portion of the payment held for the healthcare provider may be held in an escrow account. Payment may be made to the healthcare provider upon one or more agreed-upon transactional points (e.g., doses ordered, shot(s) dispensed).

Patient orders may be stored in a relational database optimized for efficient storage and retrieval. The database may consist of a series of related tables, with primary keys used to uniquely identify orders and foreign keys used to associate orders with the selected health care provider. All information related to patient orders may be stored and backed up in the database. For example, the database may be backed-up incrementally daily and backed-up fully each week. Backups may be stored off-site from the server system.

Patients may be prompted to order flu shots through the system through a marketing campaign that may include (but is not limited to) an email campaign including a link to the web site, an advocacy campaign with world health organizations that promotes the efficacy of the online reservation system, radio commercials, bill boards, and brochures and other advertising distributed at doctor offices. The campaign may encourage users to use the online system to reserve flu-shots in advance of the flu season.

The online system incentivizes user's to select a pandemic influenza option contract and also to select an annual auto-renewal of both the annual flu-shot reservation and also the annual pandemic influenza option contract. This incentivization may be in the form of price reductions for each reservation and option contract.

Patient orders may be entered in the web site and stored in the associated database. Patients may select a healthcare provider in association with their order. To enable selection of a healthcare provider, the system may provide a matching function that allows a patient to search on one or more of the following data points: healthcare provider name, healthcare provider code, zip code, city/town, state. The system may return providers that match the entered criteria. The patient may select one of the providers in order to complete the flu shot reservation, or the provider may be assigned to the patient by the system.

At an appropriate intervals (real-time, daily, weekly, monthly), order reports may be generated by the system that consolidate all order information for a given healthcare provider. Reports may include relevant patient data, quantity and order data. Reports may provide sufficient information to allow the healthcare provider to place an order with its distributor/manufacturer for the flu shots required to service the reservations received. Reports may be delivered to the healthcare provider in one of the following manners: email, fax, online password-protected extranet, or mail. Order placement with the distributor/manufacturer may be processed by the healthcare provider office, or by the system. This may depend on advance arrangements made between the health care provider office and the system.

Once orders have been delivered to the healthcare provider, an email reminder or a series of email reminders may automatically be generated and delivered to patients reminding them to call to make an appointment with the healthcare provider to receive their flu shot. The emails will include contact information for the healthcare provider, as well as a summary of the reservation that was placed with the system. If appropriate, reminders may also take the form of a telephone call from the reservation receiving center or a mailed letter.

The system may include a sign-up form for health care providers that wish to participate in the reservation system. The sign-up form may be completed by the healthcare provider or an agent of the system (in the event the agent receives a sign-up request from an interested healthcare provider via fax, phone, email or a related means). The sign-up form may seek information required for the matching node to function (primary contact, location, hours, etc.), as well as preferences related to delivery of consolidated orders to the healthcare provider (means, frequency, etc.). Healthcare providers may be approved by an agent of the system before they are available for matching on the system.

At the commencement of flu shot order season, the system may place orders with vaccine distributors/manufacturers on behalf of healthcare providers who have requested it do so. Orders may be delivered in accordance with the distributor/manufacturer preferences. In this scenario, the system may pay the distributor/manufacturer directly upon being invoiced. The doctor's administration fee, if included in the initial payment by the patient, may be released to the doctor upon receipt of an invoice for services provided by the healthcare provider to the system.

While various embodiments of the present invention are presented above, it is noted that these foregoing examples are provided merely for purposes of explanation and are not for purposes of limitation. While the present invention may be described with reference to one or more exemplary embodiments, the language used to set forth the exemplary embodiments, are words of description and not words of limitation. Although the present invention is described with reference to particular means, materials and structures, the present invention is not intended to be limited to the particulars disclosed, rather the present invention extends to all present and later-developed equivalents of those set forth herein as appreciated by one of ordinary skill in the relevant art.

What is claimed is:

1. A method for reserving a flu-shot, the method comprising:
receiving a flu-shot reservation from a user or intermediary; and
matching the reservation to a health care provider.

2. The method of claim 1, wherein the step of receiving the flu-shot reservation comprises receiving the flu-shot reservation over an internet connection.

3. The method of claim 1, wherein the user or intermediary is prompted to enter into an annual pandemic influenza vaccine option contract.

4. The method of claim 3, wherein the flu-shot reservation and the annual pandemic influenza vaccine option contract are pre-designated for automatic annual renewal.

5. The method of claim 4, wherein the step of receiving the flu-shot reservation and/or the annual pandemic influenza vaccine option contract further comprises receiving payment.

6. The method of claim 5, wherein receiving payment comprises receiving payment in a secure manner.

7. The method of claim 1, wherein the step of receiving the flu-shot reservation comprises receiving one or more of the following data parameters: (a) number of flu-shot(s), (b) name(s) of recipient(s), (c) address(es) of recipient(s), (d) health care provider preference(s), and (e) insurance provider(s).

8. The method of claim 7, wherein receiving the data parameter comprises receiving data in a secure manner.

9. The method of claim 1, further comprising a step of storing the user or intermediary's flu-shot reservation request in a reservation receiving center.

10. The method of claim 9, wherein the reservation receiving center comprises a server, a telephone call center, a mailroom, or a combination thereof.

11. The method of claim 1, further comprising a step of prompting the user or intermediary to request a flu-shot reservation.

12. The method of claim 11, wherein the step of prompting the user or intermediary to request a flu-shot reservation comprises an advertisement or solicitation.

13. The method of claim 1, further comprising a step of consolidating flu-shot reservation requests for a health care provider into a consolidated flu-shot order.

14. The method of claim 13, further comprising a step of communicating the consolidated flu-shot order to a flu-shot manufacturer or distributor.

15. The method of claim 1, further comprising a step of prompting a user or intermediary to make an appointment for flu-shot administration with the health care provider matched to said user.

16. The method of claim 14, further comprising a step of prompting a user or intermediary to make an appointment for flu-shot administration with a health care provider matched to said user.

17. The method of claim 15, wherein the step of prompting the user or intermediary to make an appointment to receive a flu-shot comprises placing a telephone call, sending an electronic mail message, sending a letter or a combination thereof.

18. The method of claim 1, wherein the step of matching the reservation to a health care provider comprises matching a data parameter of a health care provider with a data parameter of the reservation.

19. The method of claim 1, further comprising a step of communicating health care provider receipt of a flu-shot order to the user or intermediary.

20. The method of claim 16, further comprising a step of communicating health care provider receipt of a flu-shot order to the user or intermediary.

21. The method of claim 19, wherein the step of communicating health care provider receipt of the flu-shot order comprises placing a telephone call, sending an electronic mail message, sending a letter, or a combination thereof.

22. A system for reserving a flu-shot, the system comprising:
a means for receiving a flu-shot reservation from a user or intermediary; and
a means for matching the reservation to a health care provider.

23. The system of claim 22, wherein the means for receiving the flu-shot reservation comprises an internet connection.

24. The system of claim 22, wherein the user or intermediary is prompted to enter into an annual pandemic influenza vaccine option contract.

25. The system of claim 24, wherein the flu-shot reservation and the annual pandemic influenza vaccine option contract are pre-designated for automatic annual renewal.

26. The system of claim 25, wherein the means for receiving the flu-shot reservation and/or the annual pandemic influenza vaccine option contract further comprises a means for receiving payment.

27. The system of claim 26, wherein the means for receiving payment is secure.

28. The system of claim 22, wherein the means for receiving the flu-shot reservation comprises a means for receiving one or more of the following data parameters: (a) number of flu-shot(s), (b) name(s) of recipient(s), (c) address(es) of recipient(s), (d) health care provider preference(s), and (e) insurance provider(s).

29. The system of claim 28, wherein the means for receiving data parameters is secure.

30. The system of claim 22, further comprising a means for storing the user or intermediary's flu-shot reservation request in a reservation receiving center.

31. The system of claim 30, wherein the reservation receiving center comprises a server, a telephone call center, a mailroom, or a combination thereof.

32. The system of claim 22, further comprising a means for prompting the user or intermediary to request a flu-shot reservation.

33. The system of claim 32, wherein the means for prompting the user or intermediary to request a flu-shot reservation comprises an advertisement or solicitation.

34. The system of claim 22, further comprising a means for consolidating flu-shot reservation requests for a health care provider into a consolidated flu-shot order.

35. The system of claim 34, further comprising a means for communicating the consolidated flu-shot order to a flu-shot manufacturer.

36. The system of claim 35, further comprising a means for prompting a user or intermediary to make an appointment for flu-shot administration with a health care provider matched to said user.

37. The system of claim 36, wherein the means for prompting the user or intermediary to make an appointment comprises a telephone, an electronic mail, a letter, or a combination thereof.

38. The system of claim 22, wherein the means for matching the user or intermediary to the health care provider comprises a data base.

39. The system of claim 22, further comprises a means for communicating health care provider receipt of a flu-shot order to the user or intermediary.

40. The system of claim 39, wherein the means for communicating health care provider receipt of a flu-shot order to the user or intermediary comprises a telephone, an electronic mail, a letter or a combination thereof.

41. A system for reserving a flu-shot, the system comprising:
a user interface; and
a matching node for matching a user or intermediary to a health care provider.

42. The system of claim 41, wherein the user interface is capable of receiving data from the user or intermediary.

43. The system of claim 41, wherein the user or intermediary is prompted to enter into an annual pandemic influenza vaccine option contract.

44. The system of claim 43, wherein the flu-shot reservation and the annual pandemic influenza vaccine option contract are pre-designated for automatic annual renewal.

45. The system of claim 42, wherein the data can be one or more of the following: (a) number of flu-shot(s), (b) name(s) of recipients(s), (c) address(es) of recipient(s), (d) health care provider preference(s), and (e) insurance provider(s).

46. The system of claim 41, wherein the user interface comprises a website, a telephone, a mail order form, or a combination thereof.

47. The system of claim 41, wherein the user interface is capable of receiving payment from the user or intermediary.

48. The system of claim 41, wherein the matching node comprises a database of health care providers and is capable of matching a user or intermediary's data parameter to a data parameter of a healthcare provider.

49. The system of claim 41, further comprising a reservation receiving center for receiving a flu-shot reservation.

50. The system of claim 49, wherein the reservation receiving center comprises a server, a telephone call center, a mailroom, or a combination thereof.

51. The system of claim 41, further comprising a consolidation node for consolidating flu-shot reservation requests for the health care provider into the consolidated flu-shot order.

52. The system of claim 51, wherein the consolidation node comprises a database of health care providers, in communication with the matching node, to consolidate flu-shot reservations matched to a healthcare provider into a consolidated flu-shot order.

53. The system of claim 51, wherein the consolidation node is capable of sending the consolidated flu-shot order to the flu-shot manufacturer or distributor.

54. The system of claim 41, further comprising a communications network permitting communication between the user interface and the matching node.

55. The system of claim 54, wherein the communications network comprises a telephone network, a computer network, a mail delivery network, a satellite network, a wireless network, an electronic mail network, an internet connection, or a combination thereof.

56. The system of claim 54, wherein the communications network is secure.

57. The system of claim 41, wherein the system is capable of prompting the user or intermediary to make an appointment for a flu-shot by telephone, electronic mail, a letter, or a combination thereof.

58. A method of promoting vaccine production capacity expansion comprising:
soliciting annual pandemic influenza option contracts to users and intermediaries;
executing and entering into annual pandemic influenza option contracts with users and intermediaries;
collecting payments from users and intermediaries for the annual option contracts;
executing and entering into annual pandemic influenza option contracts with vaccine manufacturers
transferring a portion of the user and intermediary option contract payments to vaccine manufactures; and
wherein the option contract with vaccine manufacturer's requires vaccine an expansion of vaccine production capacity and to giving priority of distribution of vaccine in the event of a pandemic influenza outbreak to users and intermediaries holding annual pandemic influenza option contracts.
